(19) United States
(12) Patent Application Publication
(10) Pub. No.: US 2006/0281884 A1
(43) Pub. Date: Dec. 14, 2006

(54) Heavy-hydrogenated (meth) acrylates, proces for producing them, polymers thereof and optical members

(76) Inventors: Hiroki Sasaki, Kanagawa (JP); Kohzaburoh Yamada, Kanagawa (JP); Tsuneaki Maesawa, Saitama (JP); Nobuhiro Ito, Saitama (JP); Kazuhige Muto, Saitama (JP)

Correspondence Address: SUGHRUE MION, PLLC, 2100 PENNSYLVANIA AVENUE, N.W., SUITE 800, WASHINGTON, DC, 20037, US

(21) Appl. No.: 10/565,535

(22) PCT Filed: Jul. 23, 2004
(86) PCT No.: PCT/JP04/10868

§ 371 Date: Jul. 17, 2006

(30) Foreign Application Priority Data
Jul. 24, 2003 (JP) 2003-278950

(51) Int. Cl. C08F 118/02; C07C 69/52

(52) U.S. Cl. 526/282; 526/319; 560/220

(57) ABSTRACT

A novel compound represented by a formula [1] wherein R¹ and R² respectively represent a heavy or light hydrogen atom, R³ represents a heavy or light hydrogen atom or a methyl group in which three hydrogen atoms are respectively heavy or light hydrogen atoms, R⁴ represents a condensed ring group composed of a norbornane ring and a C₅₋₇ hydrocarbon ring provided that at least one hydrogen atom contained in the condensed ring group is a heavy hydrogen atom; and a novel polymer produced by polymerization of a composition comprising the compound are disclosed.

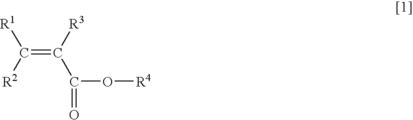

### TECHNICAL FIELD

The present invention relates to novel heavy-hydrogenated (meth)acrylates, especially relates to heavy-hydrogenated (meth)acrylates useful as material for optical fibers which are excellent in thermostability and transparency, and also relates to polymers obtained by using them.

### RELATED ARTS

Various copolymers of heavy-hydrogenated methyl methacrylate and any heavy-hydrogenated (meth)acrylate have been used as a common polymer for optical fibers, and however they still have some disadvantages of being used actually. For example, heavy-hydrogenated (meth)acrylates with a low heavy-hydrogenation content still contain many C-H bonds, and thus, polymers prepared by copolymerization of such heavy-hydrogenated (meth)acrylate and heavy-hydrogenated methyl methacrylate, which are disclosed in JPA No. syo 63-130563 (1988-130563) (the term “JPA” as used herein means an “unexamined published Japanese patent application), may have low transparency and large propagating-light loss. Accordingly, such polymers may not be suitable for material of optical fibers to be used in high-capacity and high-speed transmitting system.

For another example, polymers prepared by copolymerization of heavy-hydrogenated norbornyl (meth)acrylate and heavy-hydrogenated methyl methacrylate, which are disclosed in JPA No. syo 63-130563 (1988-130563), may not have sufficient thermostability as material for optical fibers to be used under sever conditions such as optical fibers to be used in an automotive engine.

On the other hand, it has been also tried to use polymers prepared by copolymerization of any un-heavy-hydrogenated methacrylate and heavy-hydrogenated methyl methacrylate as main polymers of a material for optical fibers. Some polymers prepared by copolymerization of heavy-hydrogenated methyl methacrylate and methacrylate having an alicyclic hydrocarbon group such as cyclohexyl methacrylate or derivatives thereof, bornyl methacrylate, phentyl methacrylate or menthyl methacrylate are disclosed in JPA No. syo 60-098407 (1985-098407) and JPA No. syo 60-125807 (1985-125807), and however such polymers may not be suitable for material of optical fibers to be used in high-capacity and high-speed transmitting system, since the methacrylates having the alicyclic hydrocarbon group have many C-H bonds and thus, they can be used properly for only limited wavelength of light source.

Polymers prepared by copolymerization of heavy-hydrogenated or un-heavy-hydrogenated adamantyl (meth)acrylate or their derivatives and heavy-hydrogenated methyl methacrylate are disclosed in JPA Nos. syo 60-125807 (1985-125807) or hei 9-235322 (1997-235322), and such polymers may be improved in thermostability to some level. However such polymers have a disadvantage for industrial use, since compounds having an adamantyl segment which is required for producing adamantyl(meth)acrylate or its derivatives as monomers for starting material of the said polymers are quite expensive. Namely, from the view of cost, the polymers, which are prepared by using such monomers, are not practical for industrial use.

Reflecting such a situation, it is required to provide heavy-hydrogenated monomers at lower cost which are useful for industrial and practical use and can form polymers by being copolymerized with heavy-hydrogenated methyl methacrylate, which can be used as a starting material for optical fibers having sufficient thermostability to be used under a sever condition such as high-temperature atmosphere, high transparency and sufficient low propagating-light loss to be used in high-capacity and high-speed transmitting system.

An object of the present invention is to provide heavy-hydrogenated(meth)acrylates with a high heavy-hydrogenation content capable of being produced at low cost industrially, and to provide polymers which can be produced by using the heavy-hydrogenated(meth)acrylates. And another object of the present invention is to provide heavy-hydrogenated monomers and polymers which can be used as a starting material for optical fibers capable of being used under a severe condition, such as a high-temperature atmosphere, with superior thermostability and transparency.

In order to achieve the objects, the present invention provides a compound represented by a formula [1]:

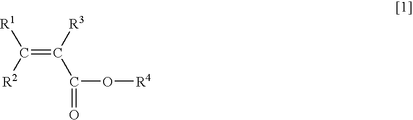

wherein R¹ and R² respectively represent a heavy or light hydrogen atom, R³ represents a heavy or light hydrogen atom or a methyl group in which three hydrogen atoms are respectively heavy or light hydrogen atoms, R⁴ represents a condensed ring group composed of a norbornane ring and a C₅₋₇ hydrocarbon ring provided that at least one hydrogen atom contained in the condensed ring group is a heavy hydrogen atom.

The C₅₋₇ hydrocarbon ring may be a saturated or unsaturated hydrocarbon ring. Examples of the saturated hydrocarbon ring include a cyclopentane ring, a cyclohexane ring and a norbornane ring; and examples of the unsaturated hydrocarbon ring include a cyclopentene ring, a cyclohexene ring and norbornene ring.

The number of heavy hydrogen atoms contained in the compound represented by the formula [1] is desirably not smaller than 20% and more desirably not smaller than 40% with respect to the total number of hydrogen atoms contained in the compound.

As embodiments of the present invention, the compound represented by the formula [1] wherein the total number of light hydrogen atoms contained in the compound is not greater than 15; the compound wherein the number of heavy hydrogen atoms contained in the R⁴ is not smaller than 10% with respect to the total number of hydrogen atoms in the R⁴, the compound wherein the total number of light hydrogen atoms contained in R⁴ is not greater than 12; and the compound wherein R⁴ is a tricyclo[5.2.1.0^{2,6}]decyl group, and at least one hydrogen atom contained in R⁴ is a heavy hydrogen atom are provided.

From another aspect, the present invention provides a polymer produced by polymerization of a composition containing the compound represented by the formula [1]; the polymer wherein 50% or more hydrogen atoms are heavy hydrogen atoms; an optical member comprising a region formed of the polymer; and the optical member which gives an absorbance at 910 nm being 50% or smaller percentage of that given by a polymer having a same structure except that all hydrogen atoms are light hydrogen atoms.

It will be noted that, in the specification, the term of “hydrogen atom” is a generic term for “light hydrogen atom” and “heavy hydrogen atom”; and the term of “heavy hydrogen atom” is used for deuterium (D) or tritium (T).

Further, in the specification, the term of “heavy-hydrogenation content” means a rate of a number of heavy hydrogen atoms to the total number of hydrogen atoms in a compound or a group.

### BRIEF DESCRIPTION OF THE DRAWINGS

FIG. 1 is a graph showing near-IR absorption spectra determined for polymer rods of Example Nos. 5 and 6 and Comparative Example No. 1

### DETAILED DESCRIPTION OF THE INVENTION

The embodiments of the present invention are described in detail bellow.

The present invention relates to compounds represented by a formula [1] below.

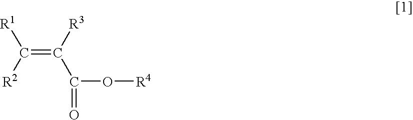

In the formula, R¹ and R² respectively represent a heavy or light hydrogen atom, R³ represents a heavy or light hydrogen atom or a methyl group in which three hydrogen atoms are respectively heavy or light hydrogen atoms, R⁴ represents a condensed ring group composed of a norbornane ring and a C₅₋₇ hydrocarbon ring provided that at least one hydrogen atom contained in the condensed ring group is a heavy hydrogen atom.

It is preferred that at least one of R¹ and R² is a heavy hydrogen atom, and more preferred that both of them are heavy hydrogen atoms.

When R³ represents a hydrogen atom, a heavy hydrogen atom is preferred; and when R³ represents a methyl group, at least one of three hydrogen atoms in the methyl group is desirably a heavy hydrogen atom, more desirably two of them are heavy hydrogen atoms and much more desirably all of them are heavy hydrogen atoms.

It is most preferred that R³ represents a heavy-hydrogenated methyl group in which all of three hydrogen atoms are heavy hydrogen atoms.

The C₅₋₇ hydrocarbon ring, which can form the condensed ring group represented by R⁴ by being condensed with a norbornane ring, may be saturated or unsaturated hydrocarbon rings, and the rings may be bridged.

Examples of the saturated hydrocarbon ring include a cyclopentane ring, a cyclohexane ring, a cycloheptane ring, a norbornane ring and a tricyclo[2.2.1.0]heptane ring. Among these, a cyclopentane ring, a cyclohexane ring and a norbornane ring are desired, and a cyclopentane ring is especially preferred.

Examples of the unsaturated hydrocarbon ring include a cyclopentene ring, a cyclopentadiene ring, a cyclohexene ring, a 1,4-cyclohexadiene ring, 1,3-cyclohexadiene ring, a norbornene ring and a 2,5-norbornanediene ring. Among these, a cyclopentene ring, a cyclohexene ring and a norbornene ring are desired, and a cyclopentene ring is especially preferred.

R⁴ desirably represents a condensed ring group formed by condensation of a norbornane ring and a saturated hydrocarbon ring, and preferred examples of such group include a tricyclo [5.2.1.0^{2,6}]decyl group, a tricyclo [6.2.1.0^{2,7}]undecyl group and a tetracyclo[6.2.1.0^{2,6}]dodecyl group. Among these, a tricyclo[5.2.1.0^{2,6}]decyl group is especially desired.

In the condensed ring group represented by R⁴ as mentioned above, at least one of hydrogen atoms included in the group is a heavy hydrogen atom, and the larger the number of heavy hydrogen atom is, the more desired it is. The ratio of a number of heavy hydrogen atoms to the total number of hydrogen atoms included in the group represented by R⁴ is desirably not less than 10%, more desirably not less than 20% and much more desirably not less than 40%. The number of light hydrogen atoms included in the group represented by R⁴ is desirably not larger than 12, more desirably not lager than 10, and much more desirably not larger than 9.

The ratio of a number of heavy hydrogen atoms to the total number of hydrogen atoms included in the compound represented by the formula [1] is desirably not less than 20%, more desirably not less than 40%, much more desirably not less than 50%, further much more desirably not less than 60% and most desirably not less than 65%. The number of light hydrogen atoms included in the compound represented by the formula [1] is desirably not larger than 15, more desirably not larger than 13, much more desirably not larger than 11 and further much more desirably not larger than 9.

The compound of the present invention represented by the formula [1] may be produced by carrying out reaction of a compound represented by a formula [2]:

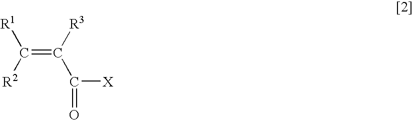

wherein X is a halogen atom, a hydroxyl group or an alkoxy group; and R¹, R² and R³ are respectively same as those in the formula [1]; and a compound represented by a formula [3]:
R⁴—OH [3]

wherein R⁴ is defined as same as that in the formula [1].

In the formula [2], examples of the halogen atom represented by X include a chlorine atom, a bromine atom, a fluorine atom and an iodine atom. Among these, a chlorine atom and a bromine atom are desired and a chlorine atom is more desired.

The alkoxy group represented by X may be linear, branched or cyclic, and is desirably selected from C₁₋₄ alkoxy groups, more desirably selected from C₁₋₂ alkoxy groups and much more desirably a C₁ alkoxy group. Examples of the alkoxy group include a methoxy group, an ethoxy group, a propyloxy group, an isopropyloxy group, a butoxy group, an isobutoxy group, a sec-butoxy group, a tert-butoxy group and a cyclopropyloxy group.

In the compound represented by the formula [2], a compound, having a larger ratio of a number of heavy hydrogen atoms to the total number of hydrogen atoms included in R¹, R² and R³, is more desired. And compounds in which all of hydrogen atoms are heavy atoms are most preferred.

It is to be noted that hydrogen atoms included in X may be light or heavy hydrogen atoms when X is a hydroxyl group or an alkoxy group.

The compounds represented by the formula [2] can be produced according to the known methods such as those described in JPA No. sho 63-130563 (1988-130563). Namely, the compound represented by the formula [2] may be produced by carrying out heavy-hydrogenation of a corresponding compound, in which all of or a part of hydrogen atoms are light hydrogen atoms, in the presence of alkaline-earth metal salt and a polymerization inhibitor (an agent for preventing polymerization) in heavy water.

The compound represented by the formula [2] may also be produced by the following method. The same compound as acrylic acid or acid halide thereof, in which all of the hydrogen atoms are light hydrogen atoms, except that a carbon-carbon double bond is replaced a carbon-carbon triple bond, is heavy-hydrogenated in the presence of calcium hydroxide in heavy-hydrogenated solvent such as heavy water, thereby a light hydrogen atom which bonds a carbon atom forming a triple bond is heavy-hydrogenated, and then the obtained compound is reacted with a heavy-hydrogen gas in the presence of Lindlar catalyst, thereby the triple bond in the obtained compound is reduced to a double bound and all light hydrogen atoms which bond carbon atoms forming a double bond are heavy-hydrogenated. According to the above method, the compounds represented by the formula [2], in which all of hydrogen atoms contained in R¹, R² and R³ are heavy hydrogen atoms, can be obtained.

A compound represented by the formula [3] may be produced by carrying out reaction of a corresponding compound, in which all or a part of hydrogen atoms in R⁴ are light hydrogen atoms, with heavy water in the presence of palladium catalyst under an atmosphere of light hydrogen gas.

A compound represented by the formula [1], in which R⁴ is a condensed ring group of a norbornane ring and a C₅₋₇ saturated hydrocarbon ring, may be produced by carrying out reaction of a compound represented by the formula [3], in which R⁴ is a condensed ring group of a norbornane ring and a C₅₋₇ saturated hydrocarbon ring, and a compound represented by the formula [2]. And further, a compound represented by the formula [1], in which R⁴ is a condensed ring group of a norbornane ring and a C₅₋₇ saturated hydrocarbon ring, may also be produced by using a compound represented by the formula [3], in which R⁴ is a condensed ring group of a norbornane ring and a C₅₋₇ unsaturated hydrocarbon ring corresponding to the saturated hydrocarbon ring, as a material, reducing the unsaturated bonds in the unsaturated ring of the compound, and then reacting the obtained compound with a compound represented by the formula [2]. It is to be noted that by using a heavy hydrogen gas for reduction of unsaturated bonds in the unsaturated ring, heavy-hydrogenation of light hydrogen atoms in R can be carried out as well as reduction of the unsaturated bonds.

In the case of using a compound represented by the formula [2] in which X is a halogen atom, a compound represented by the formula [1] may be produced by carrying out reaction of the compound represented by the formula [2] with the compound represented by the formula [3] in the presence of a suitable base, if necessary in a suitable solvent. This process will be referred to as “first embodiment” hereinafter.

According to the first embodiment, an amount of the compound represented by the formula [3] to be used is desirably from 0.8 to 1,000 times by mole, more desirably 0.8 to 100 times by mole, much more desirably from 0.8 to 50 times by mole and further much more desirably from 0.8 to 10 times by mole of the compound represented by the formula [2].

The base which can be used in the first embodiment may be selected from bases which have been used in general esterifications of acid halide and alcohol. Examples of the base include organic amines such as triethylamine, N,N-dimethylaniline, piperidine, pyridine, 4-dimethylaminopyridine, N-methylmorpholine, 1,5-diazabicyclo[4.3.0]non-5-en, 1,8-diazabicyclo[5.4.0]undec-7-en and tri-n-butylamine; and alkaline metal compounds such as sodium hydride and n-buthyllithium.

According to the first embodiment, an amount of the base to be used is desirably from 1 to 50 times by mole, more desirably from 1 to 20 times by mole and much more desirably from 1 to 5 times by mole of the compound represented by the formula [2].

Examples of the solvent to be used if necessary in the first embodiment include ethers such as diethylether, diisopropyl ether, ethyl methyl ether, tetrahydrofuran, 1,4-dioxane and dimethoxyethane; halogenated hydrocarbons such as chloromethane, methylene chloride, chloroform, carbon tetrachloride, dichloroethane, trichloroethane and chlorobenzene; hydrocarbons such as n-hexane, benzene, toluene and xylene; esters such as ethyl acetate, butyl acetate and methyl propionate; nitrites such as acetonitrile; amides such as N,N-dimethylformamide, etc. These solvents may be used alone, or in a proper combination of two or more kinds thereof.

According to the first embodiment, a volume of the solvent to be used is desirably from 0 to 100 times, more desirably from 0 to 50 times and much more desirably from 0 to 20 times with respect to the volume of the compound represented by the formula [2].

According to the first embodiment, a reaction temperature is desirably from −20 to 200° C., more desirably from −20 to 100° C. and much more desirably from −10 to 70° C. A reaction time -is desirably from 0.5 to 200 hours, more desirably from 0.5 to 36 hours and much more desirably from 0.5 to 12 hours.

In the case of using a compound represented by the formula [2] in which X is a hydroxyl group, a compound represented by the formula [1] may also be produced by carrying out reaction of the compound represented by the formula [2] and the compound represented by the formula [3] in the presence of a suitable dehydration condensing agent or a suitable acid catalyst, if necessary in a suitable solvent. The former process, in which the dehydration condensing agent is used, is referred to as “second embodiment” hereinafter; and the latter process, in which the acid catalyst is used, is referred to as “third embodiment” hereinafter.

According to the second or third embodiment, an amount of the compound represented by the formula [3] to be used is desirably from 0.8 to 1,000 times by mole, more desirably from 0.8 to 100 times by mole, much more desirably from 0.8 to 50 times by mole and further much more desirably from 0. 8 to 10 times by mole of the compound represented by the formula [2].

According to the second embodiment, the dehydration condensing agent may be selected from agents used in general dehydration condensing reactions. Examples of the dehydration condensing agent which can be used in the second embodiment include inorganic dehydration agents such as diphosphorus pentaoxide and zinc chloride anhydride; carbodiimides such as dicyclohexylcarbodiimide, diisopropylcarbodiimide and 1-ethyl-3-(3-dimethylaminopropylcarbodiimide)hydrochloride; polyphosphoric acid, acetic anhydride, carbonyl diimidazole, p-toluenesulfonylchloride, etc. An amount of the dehydration condensing agent to be used is desirably from 1 to 50 times by mole, more desirably from 1 to 30 times by mole and much more desirably from 1 to 10 times by mole of the compound represented by the formula [3].

Examples of the solvent to be used if necessary in the second embodiment include ethers such as diethylether, diisopropyl ether, ethyl methyl ether, tetrahydrofuran, 1,4-dioxane and dimethoxyethane; ketones such as acetone, dimethyl ketone, ethyl methyl ketone, diethyl ketone, 2-hexanone, t-butyl methyl ketone, cyclopentanone and cyclohexanone;

halogenated hydrocarbons such as chloromethane, methylene chloride, chloroform, carbon tetrachloride, dichloroethane, trichloroethane and chlorobenzene; hydrocarbons such as n-hexane, benzene, toluene and xylene; esters such as ethyl acetate, butyl acetate and methyl propionate; nitriles such as acetonitrile; amides such as N,N-dimethylformamide, etc. These solvents may be used alone or in a proper combination of two or more kinds thereof.

According to the second embodiment, a volume of the solvent to be used is desirably from 0 to 100 times, more desirably from 0 to 50 times and much more desirably from 0 to 20 times with respect to the volume of the compound represented by the formula [2].

According to the second embodiment, a reaction temperature is desirably from −20 to 100° C., more desirably from −20 to 80° C. and much more desirably from −10 to 50° C. And a reaction time is desirably from 0.5 to 200 hours, more desirably from 0.5 to 36 hours and much more desirably from 0.5 to 12 hours.

Examples of the acid catalyst to be used in the third embodiment include mineral acids such as hydrochloric acid, sulfuric acid and phosphoric anhydride; organic acids such as p-toluene sulfonic acid and ethane sulfonic acid; Lewis acids such as borontrifluoride etherate, etc.

According to the third embodiment, an amount of the acid catalyst is desirably from 0.01 to 0.5 times by mole, more desirably from 0.01 to 0.2 times by mole and much more desirably from 0.01 to 0.1 times by mole of the compound represented by the formula [2].

Examples of the solvent to be used if necessary in the third embodiment include ethers such as diethylether, diisopropyl ether, ethyl methyl ether, tetrahydrofuran, 1,4-dioxane and dimethoxyethane; halogenated hydrocarbons such as chloromethane, methylene chloride, chloroform, carbon tetrachloride, dichloroethane, trichloroethane and chlorobenzene; hydrocarbons such as n-hexane, benzene, toluene and xylene, etc. These solvents may be used alone or in a proper combination of two or more kinds thereof.

According to the third embodiment, a volume of the solvent to be used is desirably from 0 to 100 times, more desirably from 0 to 50 times and much more desirably from 0 to 20 times with respect to the volume of the compound represented by the formula [2].

According to the third embodiment, a reaction temperature is desirably from 0 to 200° C., more desirably from 20 to 200° C. and much more desirably from 20 to 150° C. And a reaction time is desirably from 0.5 to 200 hours, more desirably from 0.5 to 36 hours and much more desirably from 0.5 to 12 hours.

In the case of using a compound represented by the formula [2] in which X is an alkoxy group, a compound represented by the formula [1] may also be produced by carrying out ester exchange reaction of the compound represented by the formula [2] with a compound represented by the formula [3] in the presence of a suitable acid or base catalyst, if necessary in a suitable solvent. This process is referred to as “fourth embodiment” hereinafter.

According to the fourth embodiment, an amount of the compound represented by the formula [3] is desirably from 0.8 to 1.000 times by mole, more desirably from 0.8 to 100 times by mole, much more desirably from 0.8 to 50 times by mole and further much more desirably from 0.8 to 10 by mole of the compound represented by the formula [2].

According to the fourth embodiment, the acid or base catalyst may be selected respectively from acid or base catalysts which have been used in general ester exchange reactions of ester and alcohol. Examples of the acid catalysts include sulfuric acid and p-toluene sulfonic acid; and examples of the base catalyst include potassium t-butoxide and sodium methoxide.

Examples of the solvent to be used if necessary in the fourth embodiment include ethers such as diethylether, diisopropyl ether, ethyl methyl ether, tetrahydrofuran, 1,4-dioxane and dimethoxyethane; halogenated hydrocarbons such as chloromethane, methylene chloride, chloroform, carbon tetrachloride, dichloroethane, trichloroethane and chlorobenzene; hydrocarbons such as n-hexane, benzene, toluene and xylene; nitriles such as acetonitrile; amides such as N,N-dimethylformamide, etc. These solvents may be used alone or in a proper combination of two or more kinds thereof.

According to the fourth embodiment, a volume of the solvent to be used is desirably from 0 to 100 times, more desirably from 0 to 50 times and much more desirably from 0 to 20 times with respect to the volume of the compound represented by the formula [2].

According to the fourth embodiment, a reaction temperature is desirably from 0 to 200° C., more desirably from 20 to 200° C. and much more desirably from 20 to 150° C. And a reaction time is desirably from 0.5 to 200 hours, more desirably from 0.5 to 36 hours and much more desirably from 0.5 to 12 hours.

In any reactions of the compound represented by the formula [2] and the compound represented by the formula [3] as mentioned above including the first to fourth embodiments, a polymerization inhibitor is preferably used for preventing the obtained compound represented by the formula [1] from polymerizing when the compound is purified from the reaction solution, since the compound represented by the formula [1] contains a polymerizable double bond in its molecular structure.

The polymerization inhibitor may be selected from agents which have been generally used as a polymerization inhibitor (an agent for preventing polymerization). Examples of the polymerization inhibitor include phenol type compounds such as p-methoxy phenol, t-butyl catechol, butyl hydroxy toluene and tetrakis [methylene 3-(3,5-di-tert-butyl-4-hydroxyphenyl)propionate)methane (trade name: Irganox 1010); hydroquinone type compounds such as hydroquinone, 2,5-bis(1,1,3,3-tetramethylbutyl)hydroquinone and 2,5-bis (1,1-dimethylbutyl)hydroquinone; nitrosamine type compounds such as N-nitrosophenyl hydroxylamine and N-nitrosophenyl hydroxylamine aluminum salt; inorganic salts such as lithium bromide, etc. It is to be noted that an amount of the polymerization inhibitor to be used is desirably from 10 to 10,000 ppm and more desirably from 100 to 500 ppm with respect to the weight of the compound represented by the formula [1] to be obtained. When the compound of the present invention is used in production of optical fibers, it is afraid that the residue of polymerization inhibitor might cause worsening of light loss, especially light loss accompanied staining at a high temperature. The polymerization inhibitor such as tetrakis [methylene 3-(3,5-di-tert-butyl-4-hydroxyphenyl)propionate]methane, which can be removed by a known method such as distillation or adsorption on columns, is used desirably. An amount of the residue in terms of mass is desirably not greater than 50 ppm, more desirably not greater than 10 ppm and much more desirably not greater than 5 ppm as a standard for removing.

A homopolymer or a copolymer can be easily produced by carrying out polymerization of the compound, represented by the formula [1], of the present invention alone or with another monomer, since the compound of the present invention contains a polymerizable double bond in its molecular structure. Additives such as a polymerization initiator and a chain-transfer agent may be added to a reaction system of the polymerization reaction mentioned above in order to control polymerization conditions or properties of the polymer to be obtained. In this specification, the compound represented by the formula [1] itself or a mixture of the compound and the additives are called a polymerization composition.

In the compound of the present invention represented by the formula [1], not only all of or a part of hydrogen atoms in the groups represented by R¹, R² and R³ but also all of or a part of hydrogen atoms in the group represented by R⁴ are heavy-hydrogenated, and therefore, the polymer which can be obtained by polymerization of the compound of the present invention represented by the formula [1] has an excellent in transparency for a specific wavelength compared with a polymer obtained by polymerization of a corresponding compound in which all of hydrogen atoms in the group represented by R⁴ are light hydrogen atoms. As a rate of a number of heavy hydrogen atoms to the total number of hydrogen atoms (heavy-hydrogenation content) of the compound represented by the formula [1] gets higher, the transparency of a polymer obtained by polymerization of the compound for a specific wavelength is improved. It is to be noted that, in the specification, the term of “heavy-hydrogenation content” means a rate of a number of heavy hydrogen atoms to the total number of hydrogen atoms of any compound represented by the formula [1] or any polymer which can be obtained by polymerization of any compound represented by the formula [1]. The heavy-hydrogenation content of 0% means that all of hydrogen atoms contained in a compound or a polymer are light hydrogen atoms. And a natural abandance of heavy hydrogen atom is 0.015%, and therefore a heavy-hydrogenation content of any un-heavy-hydrogenated compound can be equated with 0%.

A glass transition temperature (occasionally abbreviated to “Tg”) of the polymers obtained by polymerization of one or more kinds of the compound represented by the formula [1] is generally from 150 to 180° C. and more desirably from 170 to 180° C. in the case of heat-resistance of the polymer is required.

The compound represented by the formula [1], occasionally referred to as “the compound of the present invention”, has a glass transition temperature (Tg) equal to or higher than that of a known methacrylate having a heavy-hydrogenated adamantyl group, but it can be produced in quite lower cost compared with the known methacrylate having a heavy-hydrogenated adamantyl group because of a reasonable price of a raw material for the compound of the present invention. And therefore, the compounds of the present invention are useful for industrial use.

The compounds of the present invention are useful as a labeled compound which can be used in various studies such as reaction mechanism studies and metabolic studies. And polymers obtained by polymerization of the compound of the present invention or by copolymerization of the compound of the present invention and another monomer are useful for various products such as optical members, resist materials and optical recording mediums, and are also useful for transparent products, since they are excellent in transparency. Examples of the optical member which can be produced by using the compound of the present invention include light guide elements, lenses for still cameras, video cameras, telescopes, glasses, contact lenses or solar collectors, concave mirrors, etc. Preferred examples are light guide elements and lenses.

Taking optical fibers as one example, optical members, which are embodiments of the present invention, will be described hereinafter. One embodiment of the present invention relates to an optical fiber comprising a region formed of a polymer which is produced by using the compound of the present invention. The scope of this embodiment includes optical fibers comprising the region which has a uniform refractive index and the optical fiber comprising the region which has a graded refractive index. Optical fibers are classified according to the refractive index profile, into a so-called step index type plastic optical fiber (SI type POF), a so-called multi step index type plastic optical fiber (MSI type POF) or a so-called graded index type plastic optical fiber (GI type POF), and the compound of the present invention can be used for producing any types of optical fibers. Among these, from the viewpoint of optical fiber bandwidth, GI type POF is most preferred. It is known that the MSI type or GI type refractive index can be created by adding a dopant or by combining plural polymers having a different refractive index each other.

Being used for producing of optical members, especially optical fibers, transparent polymers are preferred. It is to be noted that, in the specification, the term of “transparent polymer” is used for any polymers having a transparency not less than 50%, desirably not less than 70% and much more desirably not less than 90%, against light. When the optical member is always used at a specific wavelength range, the polymer is not required to have a high transparency at all wavelengths.

The optical fibers formed of homopolymers or copolymers of the compound represented by the formula [1] are reduced in an absorption at about 910 nm attributed to the fourth overtone of C-H stretch vibration remarkably. In the case of the optical fibers used with a commercially available light of 8 50 nm (VCSEL), the bottom of the absorption at 910 nm may have an effect on transmitting light loss. The absorption at 910 nm of the optical fiber, which is formed of the above polymer, is significantly small, the effect of the absorption is small, and the optical fiber is thus reduced in transmitting light loss. It is preferred that absorption at 910 nm of the polymer is 50% or less of that of an un-heavy-hydrogenated polymer, or in other words a polymer having zero percentage heavy-hydrogenation.

The transparent polymers, which can be used as a material for optical fibers, can be produced by polymerization of the compound of the present invention alone, or copolymerization of the compound of the present invention and at least one unsaturated ethylene monomer. Examples of the unsaturated ethylene monomer, which can be copolymerized with the compound of the present invention, include acrylates, methacrylates, acrylamides, methacrylamides, maleimides, vinyl esters, vinyl ketones, allyl compounds, olefin acids, vinyl ethers, N-vinyl amides, vinyl hetero-ring compounds, maleates, itaconates, fumarates and crotonates. Among these, (meth)acrylates are preferred, methacrylates are more preferred and methyl methacrylate is especially preferred. Form the viewpoint of transparency of the polymer, it is preferred that these unsaturated ethylene monomers are heavy-hydrogenated. The copolymer, which can be produced by copolymerization of the compound of the present invention and heavy-hydrogenated methyl methacrylate, is extremely useful as material for optical fibers. The adequate copolymerization ratio may be decided in consideration of desired properties or types of monomers to be used.

When the transparent polymers are produced, the known polymerization initiators or the know chain transfer agents may be used in order to control the molecular weight of the polymer depending on types of target optical fibers. Preferred examples of the polymerization initiator and the chain transfer agent are described in International publication WO03/19252, and examples of them include peroxide compounds and azo compounds such as 2,2′-azobisisobutyonitrile, 2,2′-azobis(2-methylbutylonitrile), 1,1′-azobis(cyclohexane-1-carbonitrile), 2,2′-azobis(2-methylpropane), 2,2′-azobis(2-methylbutane), 2,2′-azobis (2-methylpentane), 2,2′-azobis(2,3-dimethylbutane), 2,2′-azobis(2-methylhexane), 2,2′-azobis(2,4-dimethylpentane), 2,2′-azobis(2,3,3-trimethylbutane), 2,2′-azobis(2,4,4-trimethylpentane), 3,3′-azobis(3-methylpentane), 3,3′-azobis(3-methylhexane), 3,3′-azobis(3,4-dimethylpentane), 3,3′-azobis(3-ethylpentane), dimethyl-2,2′-azobis(2-methylpropionate), diethyl-2,2′-azobis(2-methylpropionate) or di-tert-butyl-2,2′-azobis(2-methylpropionate). The polymerization initiators which can be used are not limited to these, and two or more polymerization initiators may be used in combination. For satisfying requirements for various properties such as mechanical properties or transparency, the molecular weight of the polymer desirably falls within a range from 10,000 to 1,000,000. Polymerization of the compound of the present invention may be carried out according to various known polymerization methods such as solution polymerization, dispersion polymerization, bulk polymerization or emulsion polymerization, and from viewpoint of transparency, bulk polymerization is preferred. And the refractive index of the polymer may be controlled by addition of an agent for controlling refractive index, and according to a so-called interfacial gel polymerization, a refractive index profile varying along a desired direction can be created.

An ingredient for controlling refractive index is an ingredient which can give a higher refractive index in a polymer formed of a polymerizable composition when the ingredient is contained in the composition, compared with when the ingredient is not contained in the composition. The ingredient may be selected from high or low molecular weight compounds. The difference in refractive index, which is brought by addition of the ingredient, is desirably not less than 0.005. The ingredient such that a polymer containing the ingredient has a higher refractive index compared with a polymer not containing is preferred. The ingredient may be selected from polymerizable compounds. When an ingredient for controlling refractive index is polymerizable, it is preferred that the ingredient is selected from compounds which can give a higher refractive index to a copolymer containing the compound as a copolymer ingredient compared with a polymer not containing the compound. Any compounds which have the above mentioned properties, coexisting with a polymer and being stable under a polymerization condition of the compound of the present invention (such as a heating or pressurizing condition) can be used as an ingredient for controlling refractive index. Addition of the ingredient to the polymer enables the polymer to have the adequate value or the adequate profile of the refractive index depending on the application or the purpose of the polymer. For example, according to the method described in International publication WO03/19252, JPA No. 2003-75656, JPA No. 2003-149463 or the like, a core in which the refractive index is graded, can be produced by addition of the ingredient, and thus a GI-type plastic optical fiber, having a wide bandwidth, can be obtained.

Examples of the ingredient for controlling refractive index include low-molecular compounds such as benzyl benzoate (BEN), diphenyl sulfide (DPS), triphenyl phosphate (TPP), benzyl-n-butyl phthalate (BBP), diphenyl phthalate (DPP), biphenyl (DP), diphenylmethane (DPM), tricresyl phosphate (TCP) or diphenyl sulfoxide (DPSO). Among these, particularly preferable species are BEN, DPS, TPP and DPSO. Examples of the ingredient for controlling refractive index, which can polymerize with a compound represented by the formula [1], include benzyl methacrylate, phenyl methacrylate and bromophenyl methacrylate. In the present invention, hydrogen atoms in the ingredient are desirably replaced with heavy hydrogen atoms. For example, heavy-hydrogenated bromobenzene can be used for the purpose of improvement of transparency at wide wavelengths.

The refractive index may be set to a desired value by adjusting the concentration or the distribution of the ingredient, and the types or the additional amount of the ingredient may be decided depending on the applications. Tow or more types of compounds may be used as an ingredient for controlling refractive index.

The optical material or the optical member of the present invention may be produced by the various known method such as injection molding, compression molding, micro-molding, floating molding, injection compression molding or cast molding. The various properties, such as moisture resistance, optical properties, chemical resistance, wear resistance or antifogging property, of the molded product may be improved by applying any coating treatment to the surface of the molded product.

### EXAMPLES

The present invention will specifically be described referring to the specific examples. It is to be noted that any materials, reagents, ratio of use, operations and so forth can be properly altered without departing from the spirit of the present invention. The scope of the present invention is therefore by no means limited to the specific examples shown below.

### Referential Example No. 1

### Deuteration of tricyclo 5.2.1.0^{2,6}]dec-3-en-8-ol

In 765 ml of deuterium oxide (D₂O) were suspended 45 g of tricyclo [5.2.1.0^{2,6}]dec-3-en-8-ol and 22.5 g of palladium carbon (Pd 10%), and the atmosphere of the reaction system was replaced with hydrogen gas, followed by reacting for 48 hours at 180° C. in an oil bath. After the reaction was completed, n-hexane was added to the reaction solution, and then the catalyst was removed by filtration. After that, the filtrate was separated into two liquid layers. A solvent of the obtained organic layer was evaporated under reduced pressure to give 40.7 g of deuteratedtricyclo[5.2.1.0^{2,6}]dec-3-en-8-ol in a 90% yield. The structural analysis of the obtained deuterated compound was carried out by ¹H-NMR and ²H-NMR measurements, and revealed that the average deuteration content of the obtained deuterated compound was 45%.

### Referential Example No. 2

### Deuteration of tricyclo [5.2.1.0^{2,6}]dec-3-en-8-ol

In 200 ml of methanol were suspended 2 g of palladium carbon (Pd 5%) in a stream of nitrogen anda solution of 39.1 g of tricyclo [5.2.1.0^{2,6}]dec-3-en-8-ol, which was obtained by Referential Example No. 1, dissolved in 200 ml of methanol was added to the suspension. The atmosphere of the reaction system was replaced with deuterium gas, and then the solution was reacted for 24 hours at room temperature while deuterium gas was introduced to the reaction system. After the reaction was completed, the reaction solution was filtrated, and then the filtrate was evaporated to dryness under reduced pressure to give 38.8 g of deuterated tricyclo[5.2.1.0^{2,6}]dec-3-en-8-ol in a 98% yield. The structural analysis of the obtained deuterated compound was carried out by ¹H-NMR and ²H-NMR measurements, and revealed that the average deuteration content of the obtained deuterated compound was 52%.

### Referential Example No. 3

### Deuteration of tricyclo [5.2.1.0^{2,6}]decane-8-ol

In 340 ml of deuterium oxide (D₂O) were suspended 20 g of tricyclo [5.2.1.0^{2,6}]decane-8-ol and 4.0 g of palladium carbon (Pd 10%), and the atmosphere of the reaction system was replaced with hydrogen, followed by reacting for 24 hours at 180° C. in an oil bath. After the reaction was completed, n-hexane was added to the reaction solution, and then the catalyst was removed by filtration. After that, the filtrate was separated into two liquid layers. A solvent of the obtained organic layer was evaporated under reduced pressure to give 12.1 g of deuterated tricyclo [5.2.1.0^{2,6}]decane-8-ol in a 61% yield. The structural analysis of the obtained deuterated compound was carried out by ¹H-NMR and ²H-NMR measurements, and revealed that the average deuteration content of the obtained deuterated compound was 56%.

### Example No. 1

### Synthesis of a Compound of the Present Invention

In 120 ml of dichloromethane were dissolved 38.1 g of deuterated tricyclo [5.2.1.0^{2,6}]decane-8-ol which was obtained by Referential Example No. 2, and 25.3 g of triethylamine, and to the solution, 28.8 g of methacryloyl chloride, in which all hydrogen atoms were replaced with deuterium atoms, was added dropwise under cooling with ices, and the solution was then reacted for two hours at room temperature. After the reaction was completed, the crystals precipitated in the reaction solution were removed by filtration, and p-methoxyphenol was added to the obtained filtrate, followed by distillation under reduced pressure to give 37.8 g of deuterated tricyclo[5.2.1.0^{2,6}]decane methacrylate in a 67% yield as a colorless oil having a boiling point of 103 to 108° C./2 Torr. The structural analysis of the obtained deuterated compound was carried out by ¹H-NMR and ²H-NMR measurements, and revealed that the average deuteration content of the whole obtained compound was 65.7%. The compound is referred to as “WDM-6” hereinafter.

### Example No. 2

### Synthesis of a Homopolymer

To 1.0 g of WDM-6 obtained by Example No. 1 was added 1 mg of dimethyl 2,2′-azobis(2-methylpropionate) (manufactured by Wako Pure Chemical Industries, Ltd.; trade name “V-601”) followed by polymerization for six hours under vacuum at 70° C. After the polymerization was completed, acetonitrile was then added to the reaction solution to give precipitates. The precipitates were separated from the solution by filtration and dried under reduced pressure to give 0.7 g of deuterated poly(tricyclo[5.2.1.0^{2,6}]decane methacrylate) as white powder. The obtained polymer had a glass transition temperature of about 175° C.

### Example No. 3

### Synthesis of a Copolymer

With 1.0 g of WDM-6 obtained by Example No. 1 was mixed 5.0 g of deuterated methyl methacrylate (MMA-d8), and 1 mg of dimethyl 2,2′-azobis(2-methylpropionate) (manufactured by Wako Pure Chemical Industries, Ltd.; trade name “V-601”) and 1.5 mg of laurylmercaptan were added thereto followed by polymerization for six hours under vacuum at 70° C. After the polymerization was completed, methanol was then added to the reaction solution to give precipitates. The precipitates were separated from the solution by filtration and dried under reduced pressure to give 5.6 g of deuterated poly(tricyclo[5.2.1.0^{2,6}]decane methacrylate/methyl methacrylate) as white powder. The obtained polymer had a weight-average molecular weight of 95,000 and a molecular weight distribution of 2.1. And the obtained polymer had a glass transition temperature of 125° C.

### Example Nos. 4 to 6

Three kinds of monomer, one of which contained WDM-6 (the total deuteration content 65.7%, the side-chain deuteration content 56%) alone, and others contained WDM-6 and deuterated methyl methacrylate (MMA-d8) in a weight ratio of 25/75 and 50/50 respectively, were prepared. And dimethyl-2,2′-azobis(2-methylpropionate) of 0.14 wt % with respect to the weight of the monomer as a polymerization initiator and n-laurylmercaptan of 0.2 wt % with respect to the weight of the monomer were then added to the monomers respectively to form a polymerizable composition. After being deaerated for 5 minutes with a stream of nitrogen, the polymerizable compositions were polymerized respectively for 24 hours at 70° C., 24 hours at 90° C. and further for 3 hours at 105° C. without being exposed to air to form a polymer rod.

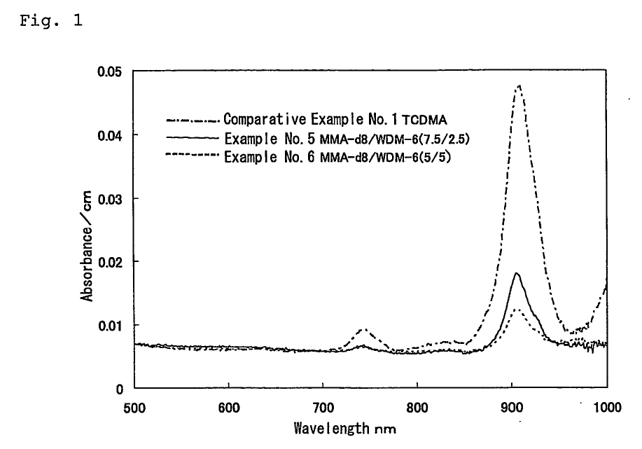

Each of the obtained polymer rods had both end portions cut off by a diamond cutter, and had cut surfaces polished optically. They are referred to as Example No. 4, 5 and 6 respectively. The near IR absorption spectra of Example Nos. 5 and 6 were determined respectively. The results were shown in FIG. 1.

### Comparative Example No. 1

Tricyclo[5.2.1.0^{2,6}]decane methacrylate (the total heavy-hydrogenation content 0%; referred to as “TCDMA” hereinafter), dimethyl-2,2′-azobis(2-methylpropionate) of 0.14 wt % with respect to the weight of TCDMA as a polymerization initiator, and n-laurylmercaptan of 0.2 wt % with respect to the weight of TCDMA as a chain transfer agent were mixed to form a polymerizable composition. After being deaerated for 5 minutes with a stream of nitrogen, the polymerizable composition was polymerized for 24 hours at 70° C., 24 hours at 90° C. and further for 3 hours at 105° C. without being exposed to air to form a polymer rod.

The obtained polymer rod had both end portions cut off by a diamond cutter, and had cut surface polished optically. This is referred to as Comparative Example No. 1. The near IR absorption spectrum of Comparative Example No. 1 was measured. The result was also shown in FIG. 1.

The results shown in FIG. 1 reveals that the polymer rods, which were respectively made of a copolymer of WDM-6 falling within the scope of the present invention and heavy-hydrogenated methyl methacrylate, had a much smaller absorption attributed to the fourth C-H overtone at about 730 nm or about 910 nm compared with the polymer rod which was prepared by polymerization of a compound having a 0% heavy-hydrogenation content. The results suggests that when an optical fiber to propagate light emitted from a commercially available 850 nm-light source is produced by using a copolymer of WDM-6 and heavy-hydrogenated methyl methacrylate as material, the optical fiber exhibits a high transparency and low propagating-light loss because the bottom of the absorption band at 910 nm has little influence on propagating-light loss. Furthermore such an optical fiber has a high glass transition temperature and thus has a high thermostability. Accordingly, it is possible to produce transparent optical fibers having high thermostability and very low absorption attributed to C-H high frequency stretching by drawing the polymer into fiber in the same manner described in International publication WO03/019252 or the like.

### INDUSTRIAL APPLICABILITY

According to the present invention, it is possible to provide heavy-hydrogenated compounds with a high heavy-hydrogenation content, which can be produced from inexpensive starting material, and thus capable of being applied to industrial uses with advantage of cost. Being copolymerized with another monomer selected from various monomer groups, the heavy-hydrogenated compounds of the present invention can form polymers having a high thermostability sufficient to be used even under severe conditions such as a high-temperature atmosphere, which can be used as a starting materials for optical fibers having a high transparency, and low propagating-light loss to be used in high-capacity and high-speed transmitting systems.

1. A compound represented by a formula [1]: 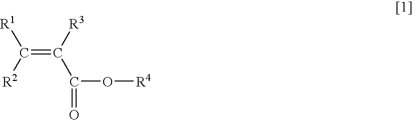
wherein R¹ and R² respectively represent a heavy or light hydrogen atom, R³represents a heavy or light hydrogen atom or a methyl group in which three hydrogen atoms are respectively heavy or light hydrogen atoms, R⁴ represents a condensed ring group composed of a norbornane ring and a C₅₋₇ hydrocarbon ring provided that at least one hydrogen atom contained in the condensed ring group is a heavy hydrogen atom.

2. The compound of claim 1, wherein the C₅₋₇ hydrocarbon ring is a saturated hydrocarbon ring.

3. The compound of claim 1, wherein the C₅₋₇ hydrocarbon ring is an unsaturated hydrocarbon ring.

4. The compound of claim 2, wherein the saturated hydrocarbon ring is selected from the group consisting of a cyclopentane ring, a cyclohexane ring and a norbornane ring.

5. The compound of claim 3, wherein the unsaturated hydrocarbon ring is selected from the group consisting of a cyclopentene ring, a cyclohexene ring and a norbornene ring.

6. The compound of claim 1, wherein 20% or more hydrogen atoms contained in the compound are heavy hydrogen atoms.

7. The compound of claim 1, wherein 40% or more hydrogen atoms contained in the compound are heavy hydrogen atoms.

8. The compound of claim 1, wherein the total number of light hydrogen atoms contained in the compound is not greater than 15.

9. The compound of claim 1, wherein 10% or more hydrogen atoms contained in R⁴ are heavy hydrogen atoms.

10. The compound of claim 1, wherein the total number of light hydrogen atoms contained in R⁴ is not greater than 12.

11. The compound of claim 1, wherein R⁴ is a tricyclo[5.2.1.0^{2,6}]decyl group, and at least one hydrogen atom contained in R⁴ is a heavy hydrogen atom.

12. A process for producing a compound represented by a formula [1]: 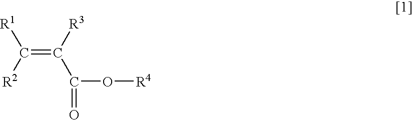
wherein R¹ and R² respectively represent a heavy or light hydrogen atom, R³ represents a heavy or light hydrogen atom or a methyl group in which three hydrogen atoms are respectively heavy or light hydrogen atoms, R⁴ represents a condensed ring group composed of a norbornane ring and a C₅₋₇ hydrocarbon ring provided that at least one hydrogen atom contained in the condensed ring group is a heavy hydrogen atom;
comprising reacting an alcohol having a condensed ring group, in which at least one hydrogen atom is a heavy hydrogen atom, composed of a norbornane ring and a C₅₋₇ hydrocarbon ring, with a compound represented by a formula [2]: 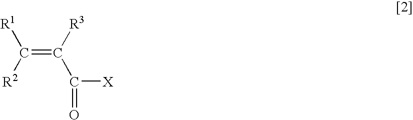
wherein R¹ and R² respectively represent a heavy or light hydrogen atom, R³ represents a heavy or light hydrogen atom or a methyl group in which three hydrogen atoms are respectively heavy or light hydrogen atoms, and X represents a halogen atom, a hydroxyl group or an alkoxy group.

13. A polymer produced by polymerization of a composition comprising the compound of claim 1.

14. The polymer of claim 13, wherein 50% or more hydrogen atoms contained in the polymer are heavy hydrogen atoms.

15. An optical member comprising a region formed of a polymer of claim 13.

16. The optical member of claim 15, which gives an absorbance at 910 nm being 50% or smaller percentage of that given by a polymer having a same structure except that all hydrogen atoms are light hydrogen atoms.
